(19) United States
(12) Patent Application Publication
(10) Pub. No.: US 2003/0013752 A1
(43) Pub. Date: Jan. 16, 2003

(54) Method for administration of cancer therapeutic

(76) Inventors: Lars Holger Breimer, Danbury, CT (US); Kapil Dhingra, Tenafly, NJ (US); Urvashi Hooda Dhingra, Cedar Grove, NJ (US); Steve Ritland, Paterson, NJ (US)

Correspondence Address: HOFFMANN-LA ROCHE INC., PATENT LAW DEPARTMENT, 340 KINGSLAND STREET, NUTLEY, NJ, 07110

(21) Appl. No.: 10/170,080

(22) Filed: Jun. 12, 2002

Related U.S. Application Data
(60) Provisional application No. 60/298,544, filed on Jun. 15, 2001.

(51) Int. Cl. A61K 31/405

(52) U.S. Cl. 514/414

(57) ABSTRACT

A method of treating a patient suffering from cancer comprising administering to the patient a compound of formula I or a therapeutically effective salt or ester thereof, in an amount from about 140 mg/m²/day to about 400 mg/m²/day for an administration period of up to about 14 days, said administration period starting on the first day of a 21-28 day treatment cycle, said treatment cycle being repeated three to four weeks for as long as the tumor remains under control and the regimen is clinically tolerated.

This application claims priority of Provisional application Serial No. 60/298,544, filed Jun. 15, 2001.

### FIELD OF THE INVENTION

The present invention is directed to methods of administration of compounds of formula I

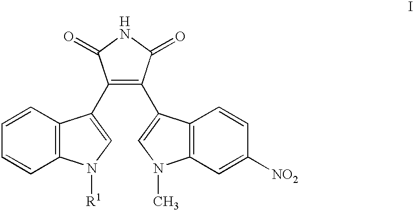

in the treatment of cancer. In particular, the invention is directed to improved methods of administration of compounds of formula I that provide desirable anti neoplastic effects with tolerable levels toxicity. The process of the invention is characterized by administering frequent doses comprising relatively low concentrations of a compound of formula I. This protocol is both safer and more efficacious than administering less frequent doses of higher concentrations.

### BACKGROUND OF THE INVENTION

The compounds of formula I below belong to a new class of orally active cell-cycle inhibitors and apoptosis inducers having potent anti-cancer therapeutic activity, in particular in solid tumors such as breast, colon, lung, bladder, skin (especially melanoma), prostrate, colon, and uterine cancers. See, e.g., U.S. Pat. Nos. 5,057,614 (reissued as Re. 36,736) and 6,048,887; and Cassidy, J. et al., Phase I Clinical and Pharmacokinetic Study of the Novel Cell Cycle Inhibitor . . . ,” Abstract 731, Presented at the May 23^{rd}, 2000 Meeting of the American Society of Clinical Oncology, all of which are herein incorporated by reference.

It has now been discovered that compounds of formula I are especially effective, and best tolerated, in cancer therapy when administered in the specific doses and pursuant to the specific protocols herein described.

### SUMMARY OF THE INVENTION

The present invention relates to a method of treating a patient suffering with cancer comprising administering to the patient a compound of formula I, or a therapeutically effective salt or ester thereof, in an amount from about 140 mg/m²/day to about 400 mg/m²/day, preferably from about 190 mg/m²day to about 300 mg/m²/day, most preferably from 190 mg/m²/day to 250 mg/m²/day for an administration period of up to about 14 days, said administration period starting on the first day of a three week (21 days) to four week (28 day) treatment cycle, said treatment cycle being repeated three to four weeks for as long as the tumor remains under control and the regimen is clinically tolerated.

### DETAILED DESCRIPTION OF THE INVENTION

As used herein the term “antineoplastic” means inhibiting or preventing the development, maturation or proliferation of malignant cells.

The term “pharmaceutically acceptable ester” of a compound of formula I means a conventionally esterified compound of formula I having a carboxyl group, which esters retain the biological effectiveness and properties of the compound of formula I.

The term “pharmaceutically acceptable salt” of a compound of formula I as used herein is any conventional salt or base addition salt that retains the biological effectiveness and properties of the compound of formula I and which is formed from a suitable non-toxic organic or inorganic acid or organic or inorganic base. Preferred salts are cationic salts, for example, of alkali metals, especially sodium salts.

The term “therapeutically effective” means an amount of drug, or combination or composition, which is effective for producing a desired therapeutic effect upon administration to a patient, for example, to stem the growth, or result in the shrinkage, of a cancerous tumor.

“Therapeutic index” is a well-recognized term of art and is an important parameter in the selection of anticancer agents for clinical trial. Therapeutic Index takes into consideration the efficacy, pharmacokinitecs, metabolism and bioavailability of anticancer agents. See, e.g., J. Natl. Cancer Inst. 81(13): 988-94 (Jul. 5, 1989).

“Tumor control” means that the perpendicular diameters of measurable lesions has not increased by 25% or more from the last measurement. See, e.g., World Health Organization (“WHO”) Handbook for Reporting Results of Cancer Treatment, Geneva (1979).

“Tumor volume (in cubic millimeter)” for purposes of measuring tumor size is calculated using the ellipsoid formula:

(D×(d²))/2

where “D” represents the large diameter of the tumor, and “d” represents the small diameter.

The present invention relates to a method of treating a patient suffering with cancer comprising administering to the patient of a compound of formula I

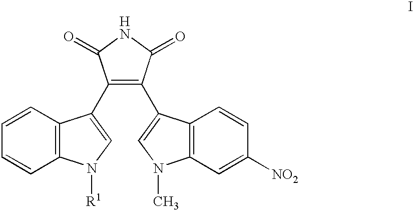

or a pharmaceutically acceptable salt said compound, wherein

R¹ is selected from the group consisting of —H, —CH₃, and —CH₂OH, in an amount from about 140 mg/m²/day to about 400 mg/m²/day, preferably from about 190 mg/m²/day to about 300 mg/m²/day, most preferably from about 190 mg/m²/day to about 250 mg/m²/day for up to about 14 days, starting on the first day of a three week (21 days) to four week (28 days) treatment cycle, said treatment cycle being repeated every 21-28 days for as long as the tumor remains under control and the regimen is clinically tolerated.

A patient's body measurement in square meters (“m^{2”}), this is a “BSA (body surface area”) measurement”, typically ranges from about 1.4 m² to about 2.2 m². Thus, the total amount of compound of formula I to be delivered in a treatment cycle (mg) is calculated as follows:

[Dose intensity(mg/m²/week)]×[BSA(m²)]×[number of weeks in treatment cycle]

In a preferred embodiment, the compound of formula I is administered daily for up to about 14 days, commencing on the first day of a treatment cycle. The course of a preferred cycle is about 21 or about 28 days, though cycles anywhere between about 21 and about 28 days are also effective and contemplated.

In a most preferred embodiment, compound of formula I is administered daily for up to about 14 days, commencing on the first day of a 28 day cycle (that is, a 4 week repeating cycle). In another preferred embodiment, compound of formula I is administered daily for up to about 7 days, commencing on the first day of a 21-28 day cycle (that is, a 3 to 4 week repeating cycle).

The compound of formula I is administered daily, as a single dose (“QD” or once daily), or divided into two or more doses daily, preferably twice per day (“BID”), most preferably twice per day at equal 12 hour intervals (“Q12”). The length of preferred treatment cycle is from about 3 to about 4 weeks.

Preferably, the compound of formula I is administered to the patient in an oral unit dosage form, more preferably in capsule or tablet form.

Preferably, four day treatment schedules are repeated every twenty one days, or as soon as permitted by recovery from toxicity, for so long as the tumor is under control or regressing and the patient tolerates the regimen.

Seven, and fourteen day treatment schedules are preferably repeated every twenty eight days. Preferably, these treatment cycles are repeated for a total of up to about eight cycles (that is a total of about twenty four or about thirty two weeks).

In an embodiment the compound of formula I is administered to a patient twice daily, preferably at equal 12 hour intervals, at a dose of about 125 mg to about 250 mg, for up to 14 consecutive days.

In a preferred embodiment, the compound of formula I is administered twice daily at equal twelve hour intervals (Q12) in an amount of from about 70 mg/m² to about 200 mg/m², preferably from about 95 mg/m² to about to 175 mg/m², more preferably from about 95 mg/m² to about 125 mg/m² for 14 consecutive days, commencing on day 1 of a 28 day cycle. This treatment is repeated every 28 days, or as soon as permitted by recovery from toxicity, for so long as the tumor is under control or regressing and the patient tolerates the regimen. Preferably, the cycles are repeated for a total of up to eight cycles (that is 32 weeks).

In another preferred embodiment the compound of formula I is also administered twice daily, preferably at equal intervals (that is “Q12”) in an amount of from about 100 mg/m² to about 280 mg/m², preferably from about 150 mg/m² to about 250 mg/m², optimally 200 mg/m², for 7 consecutive days commencing on day 1 of a 28 day cycle. This treatment is repeated every 28 days, or as soon as permitted by recovery from toxicity, for so long as the tumor is under control and the patient tolerates the regimen or tumor regression. Preferably, the cycles are repeated for a total of up to eight cycles (that is 32 weeks).

A preferred compound of formula I is:

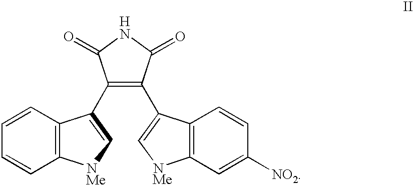

This is a known compound. See U.S. Pat. No. Re. 36,736.

Other compounds of formula I are:

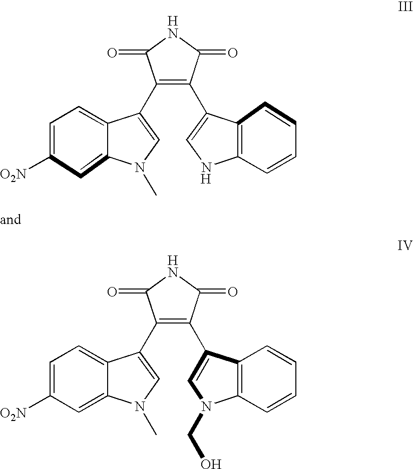

Compounds III and IV above are also known compounds. See U.S. Pat. No. 6,048,887.

The determination of tumor control (also referred to as “maintenance”) or shrinkage (also referred to as “regression”) is made by known methods. For example, by evaluation of patient symptoms, physical examination, X-ray, MRI or CAT scan or other commonly accepted evaluation modalities.

The present invention may be exemplified by controlled clinical trials as shown in the Example below, which illustrates the invention without limitation.

### EXAMPLE

Patients

Two clinical trials of the compounds of formula 1 were carried out in patients suffering from advanced, solid tumors. 85 patients were treated with a compound of formula I according to the dosage regimen of the invention. All patients had failed standard therapy of proven effectiveness for their cancer.

Drug Formulations

Hard gelatin capsules containing compound of formula II microprecipitated bulk powder (either 50% compound II and 50% Eudragit L100, or 30% compound II and 70% Eudragit) and Croscarmellose Sodium.

Treatment: Dosing Regimen

| Description of Dosing | Schedule | Daily Dose Range |
|---|---|---|
| Schedule | Abbreviation | (mg/m²) |
| Twice daily for 7 days, | q 12 h × 7, q 4 wk | 100-280 q 12 h |
| every 4 weeks | | |
| Twice daily for 14 days, | q 12 h × 14, q 4 wk | 70-150 q 12 h |
| every 4 weeks | | |

Primary Efficacy Parameter:

Patients' tumors were measured during the course of treatment by commonly accepted modalities, such as, physical examination and/or serological markers (tumor markers) and/or radiological methods such as plain X-ray, CT scan, MRI, etc. A stabilization/decrease in the size of the tumor mass or tumor marker is evidence of anticancer activity.

Results

It was unexpectedly found that approximately 20% of patients treated in accordance with the above regimen experienced stabilization or shrinkage of tumor. These included patients with a variety of solid tumors, including, tumors arising from the kidney, lung, colon, rectum, prostate, breast, pancreas, and uterus. The sites of metastatic involvement in these patients included, liver, lung, bone, lymph nodes, and skin, among others. The anticancer efficacy was observed with acceptable toxicity.

| | | | | |
|---|---|---|---|---|
| | | # Prior | Dose | |
| Age, | Tumor Type/major | Regi- | (mg/m²)/ | |
| Sex | metastatic sites | mens | Schedule | Response* |

Phase I, 7 day Schedule (cycle length 28 day)

| | | | | |
|---|---|---|---|---|
| 56, F | Unknown primary/liver | 4 | 280 Q 12 H | SD 6 + |
| | | | | cycles |
| 71, M | Prostate/regional, bone | 1 | 200 Q 12 H | SD 4 cycles |
| | mets | | | |
| 67, M | NSCLC/lung | 2 | 240 Q 12 H | PR |

Phase I, 14 day Schedule (cycle length 28 day)

| | | | | |
|---|---|---|---|---|
| 63, M | Colon/soft tissue, nodes | 2 | 70 Q 12 H | SD 6 + |
| | | | | cycles |
| 71, M | Colon/liver | 1 | 70 Q 12 H | SD 6 + |
| | | | | cycles |
| 56, M | Renal/nodal | 1 | 100 Q 12 H | SD 6 + |
| | | | | cycles |
| 57, M | Unknown primary/lung, | 3 | 100 Q 12 H | SD 7 cycles |
| | nodes | | | |
| 63, F | Endometrial | 1 | 125 Q 12 H | SD 6 + |
| | | | | cycles |
| 61, M | Renal/liver, lung, adrenal | 3 | 150 Q 12 H | SD 6 + |
| | | | | cycles |

*SD - Stable disease; PR - Partial Response

1. A method of treating a patient suffering form cancer, comprising administering to said patient a pharmaceutical composition containing as an active ingredient a compound of formula I 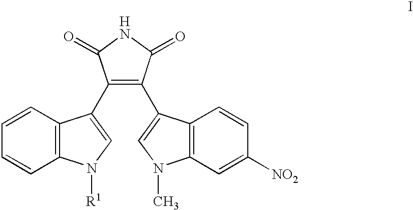
or a pharmaceutically acceptable salt or ester of said compound, wherein
R¹ is selected from the group consisting of —H, —CH₃, and —CH₂OH, in an amount from about 140 mg/m²/day to about 400 mg/m²/day for up to about 14 days, starting on the first day of a 21-28 day treatment cycle, said treatment cycle being repeated every 21-28 days.

2. The method of claim 1 wherein the compound of formula I is administered twice daily in an amount of from about 70 mg/m² to about 200 Mg/M².

3. The method of claim 2 wherein the compound of formula I is administered in equal doses at 12 hour intervals.

4. The method of claim 3 wherein the compound of formula I is administered twice daily in 12 hour intervals in an amount of from about 95 mg/m² to about 175 mg/m².

5. The method of claim 4 wherein the compound of formula I is administered twice daily in an amount of from about 95 mg/m²/Q12 to about 125 mg/m²Q12.

6. The method of claim 3 wherein the compound of formula I is administered for 14 consecutive days.

7. The method of claim 6 wherein the compound of formula I is administered commencing on day 1 of a 28 day cycle.

8. A method of treating a patient suffering form cancer, comprising administering to said patient, twice daily, a pharmaceutical composition containing as an active ingredient a compound of formula I 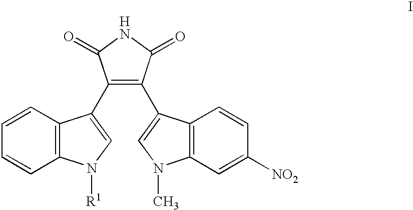
or a pharmaceutically acceptable salt or ester of said compound, wherein
R¹ is selected from the group consisting of —H, —CH₃, and —CH₂OH,
in an amount from about 125 mg to about 250 mg for up to about 14 days, starting on the first day of a 21-28 day treatment cycle, said treatment cycle being repeated every 21-28 days.

9. The method of claim 8 wherein the compound of formula I is administered in equal doses at 12 hour intervals.

10. A method of treating a patient suffering form cancer, comprising administering to said patient a pharmaceutical composition containing as an active ingredient a compound of formula I 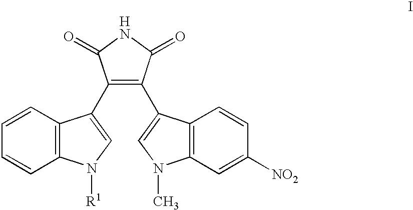
or a pharmaceutically acceptable salt or ester of said compound, wherein
R¹ is selected from the group consisting of —H, —CH₃, and —CH₂OH,
in an amount from about 190 mg/m²/day to about 250 mg/m²/day for up to about 14 days, starting on the first day of a 21-28 day treatment cycle, said treatment cycle being repeated every 21-28 days.

11. The method of claim 3, wherein the active ingredient is a compound of the formula: 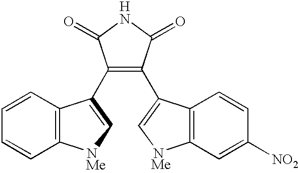
or a pharmaceutically acceptable salt or ester thereof.

12. The method of claim 3, wherein the active ingredient is a compound of the formula 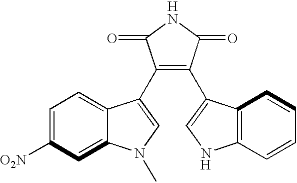
or a pharmaceutically acceptable salt or ester thereof.

13. The method of claim 11 wherein the active ingredient is a compound of the formula 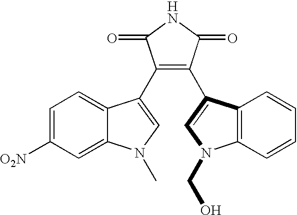
or a pharmaceutically acceptable salt or ester thereof.

14. The method of claim 11 wherein the cancer is colorectal cancer.

15. The method of claim 11 wherein the cancer is prostate cancer.

16. The method of claim 11 wherein the cancer is lung cancer.
